(19) United States
(12) Patent Application Publication
(10) Pub. No.: US 2015/0080671 A1
(43) Pub. Date: Mar. 19, 2015

(54) Sleep Spindles as Biomarker for Early Detection of Neurodegenerative Disorders

(71) Applicants: Technical University of Denmark, Kgs. Lyngby (DK); Glostrup Hospital, Glostrup (DK); H. Lundbeck A/S, Valby (DK)

(72) Inventors: Julie Anja Engelhard Christensen, Copenhagen K (DK); Lykke Kempfner, Herlev (DK); Poul Jørgen Jennum, Farum (DK); Helge Bjarup Dissing Sørensen, Graested (DK); Lars Arvastson, Malmo (SE); Søren Rahn Christensen, Vallensbaek Strand (DK)

(21) Appl. No.: 14/290,402

(22) Filed: May 29, 2014

(30) Foreign Application Priority Data
May 29, 2013 (EP) 13169679.1

(51) Int. Cl. A61B 5/00; A61B 5/11; A61B 5/0496; A61B 5/0476

(52) U.S. Cl. CPC A61B 5/4812; A61B 5/0476; A61B 5/11; A61B 5/0496; A61B 5/4082
USPC 600/301; 600/544

(57) ABSTRACT

The present invention relates to the use of sleep spindles as a novel biomarker for early diagnosis of synucleinopathies, in particular Parkinson's disease (PD). The method is based on automatic detection of sleep spindles. The method may be combined with measurements of one or more further biomarkers derived from polysomnographic recordings.

### FIELD OF INVENTION

The present invention relates to the use of sleep spindles as a novel biomarker for early diagnosis of synucleinopathies, in particular Parkinson's disease (PD). The method is based on automatic detection of sleep spindles. The method may be combined with measurements of one or more further biomarkers derived from polysomnographic recordings.

### BACKGROUND OF INVENTION

Synucleinopathies are neurodegenerative disorders characterized by Lewy bodies and include Parkinson's disease, dementia with Lewy bodies and multiple system atrophy.

Parkinson's disease (PD) is a degenerative disorder of the central nervous system. The prevalence of PD is approximately 0.5% to 1% among people 65 to 69 years of age, rising to 1% to 3% among those aged 80 years or older. The neurodegeneration occurring in PD is irreversible and there is currently no cure for the disease.

The most obvious symptoms of PD are movement-related and include unilateral tremor, rigidity, akinesia and postural instability. Later, cognitive and behavioural problems may arise, with dementia commonly occurring in the advanced stages of the disease. Other symptoms include sensory, sleep and emotional problems.

Diagnosis of PD is currently based on the clinical manifestation of the motor symptoms, and treatments are directed at managing clinical symptoms. When the diagnosis is made based on the manifestation of the motor symptoms, the brain is already severely affected as the motor symptoms of PD arise from the loss of dopamine-generating neurons in the substantia nigra.

There are currently no reliable screening techniques available, which are capable of detecting PD in its very early stages, i.e. before motor symptoms appear. Such early screening techniques could potentially lead to the identification of more efficient treatments of Parkinson's disease and possible to a cure.

Sleep spindles (SS) are bursts of oscillatory brain activity during non-REM (NREM) sleep. They can be seen as transient waveforms in electroencephalogram (EEG) derivations acquired from sleeping subjects. Sleep spindles are used for the classification of sleep stages and have been studies in connection with various psychiatric and neurological disorders.

It has recently been suggested that changes in SS have the potential to be biomarkers of some neurodegenerative diseases, such as Alzheimer's disease (Ktonas et al., 2009; Ventouras et al., 2012).

Reduced SS activity has also been reported in patients with Parkinson's disease (PD) (Comella et al., 1993).

### SUMMARY OF INVENTION

There is a need for identification of novel biomarkers for synucleinopathies allowing for an earlier detection of these diseases. Such early detection could potentially lead to the development of novel and more efficient treatments and eventually to a cure.

The present invention addresses the above problem by providing a novel biomarker allowing for early diagnosis of synucleinopathies based on automatic detection of sleep spindles. The claimed method allows for diagnosis of a synucleinopathy before the major clinical manifestations of the disease become apparent. In the case of PD, before clinical manifestation of motor symptoms. Hence, the claimed method allows for diagnosis of a synucleinopathy in a patient before substantial irreversible neurodegeneration has occurred.

In one embodiment, the present invention relates to a method for identifying a subject having an increased risk of developing a synucleinopathy comprising detection of sleep spindles.

In particular, the present invention relates to a method comprising the steps of:
a. acquiring one or more electroencephalographic (EEG) derivations from a sleeping subject,
b. detecting sleep spindles in said one or more EEG derivations, and
c. analysing the density of sleep spindles in said one or more EEG derivations,
wherein a subject having a decreased sleep spindle density has an increased risk of developing a synucleinopathy.

The sleep spindle biomarker of the present invention may be combined with measurements of one or more further biomarkers such as a biomarker based on automatic analysis of abnormal motor activity during REM sleep, a biomarker based on automatic analysis of electrooculography (EOG) signals and a biomarker based on automatic analysis of autonomic dysfunction. Combination with one or more further biomarkers can potentially increase both specificity and sensitivity of the diagnosis.

### DESCRIPTION OF DRAWINGS

FIG. 1 depicts the six Braak stages of Parkinson's disease and the clinical symptoms associated with the different stages. Currently, Parkinson's disease is diagnosed upon manifestation of motor symptoms.

FIG. 2 Method for developing the SS detector. The F3-A2 and C3-A2 EEG derivations are used for feature extraction, divided into L segments of 2 seconds with 1-second overlap. Before Matching Pursuit and feature extraction, the segments are filtered from 2 to 35 Hz. For each of the L segments, six feature values for each EEG derivation are computed. The feature matrix F of Lx12 features is used as the input for the classification step, which applies a Support Vector Machine and outputs a scalar value yl for each L segment. The sign of yl indicates whether the segment corresponds to an SS or not.

FIG. 3 Illustration of the leave-one-subject-out strategy used in this study. Each small rectangle represents a sleep epoch. Blue and white rectangles are used for testing and training, respectively. The numbers N0001-N0013 are the IDs for the control subjects. Different numbers of sleep epochs were available from each subject, so different amounts of data were held out in each run.

FIG. 4 Definition of the four variables, True Positive (TP), False Positive (FP), True Negative (TN) and False Negative (FN), based on seconds.

FIG. 5 The overall ROC curve for a mean AUC measure of 91.0%, based on the leave-one-subject-out method.

FIGS. 6A through 6C Results for N2, N3 and all NREM combined. The figures illustrate the mean and standard deviation of the individuals in the four groups and the individual measures for each subject and patient. A single asterisk indicates significant changes with p<0.05. Double asterisks indicate significant changes with p<0.01.

### DETAILED DESCRIPTION OF THE INVENTION

The present inventors have found that patients with idiopathic REM sleep behavior disorder (iRBD) have decreased sleep spindle density.

Recent research has indicated that iRBD, characterised by abnormally high muscle activity during REM sleep, may be an early marker of synucleinopathies, in particular PD. Patients suffering from iRBD are thus at high risk of developing Parkinson's disease and other synucleinopathies. In one embodiment, the present invention relates to a method for diagnosing REM sleep behaviour disorder (RBD).

### Synucleinopathies

The present invention relates in one embodiment to a method for early diagnosis of synucleinopathies, in particular Parkinson's disease. Thus, in one embodiment the present invention relates to a method for predicting the risk of a subject for developing a synucleinopathy comprising detection of sleep spindles.

In particular, the present invention relates to a method comprising the steps of:
a. acquiring one or more electroencephalographic (EEG) derivations from a sleeping subject,
b. detecting sleep spindles in said one or more EEG derivations, and
c. determining the density of sleep spindles in said one or more EEG derivations,
wherein a subject having a decreased sleep spindle density has an increased risk of developing a synucleinopathy.

The method of the present invention is preferably performed before clinical symptoms appear and precedes any major, irreversible neurodegeneration, thus allowing for very early diagnosis of a synucleinopathy. Using the method of the present invention it may thus be possible to identify patients having an increased risk of developing a synucleinopathy many years in advance of the clinical manifestation of the disease.

In a preferred embodiment detection and analysis of sleep spindles is an automated process, such as a fully automated process which does not involve or require any manual analysis of EEG recordings by a sleep expert. Manual analysis of the EEG by sleep experts is time-consuming, costly and prone to human errors. These drawbacks are avoided with the use of an automated method for detecting and analyzing sleep spindles.

According to the present invention a subject has an increased risk of developing a synucleinopathy if said subject has a decreased sleep spindle density. The sleep spindle density may e.g. be compared to the sleep spindle density in a group of healthy subjects. The healthy subjects may e.g. be a group of people who are not suffering from a synucleinopathy, iRBD or other forms of neurodegenerative disorders. The group of healthy subjects are ideally age-matched and/or gender matched.

The increased risk of developing a synucleinopathy may e.g. be at least 50%, such as at least 100%, for example at least 150%, such as at least 200% or even more compared to the risk of a comparable healthy subject of developing a synucleinopathy.

The subject itself may also be used as the control, i.e. sleep spindle density of a subject is compared to a previous measurement of sleep spindle density in the same subject. If the sleep spindle density is decreased compared to a previous measurement in the same subject, the subject has an increased risk of developing a synucleinopathy. The previous measurement is preferably obtained several years before, such as 5 years or more, for example 8 years or more, such as 10 years or more.

Sleep spindle density is defined herein as the number of detected sleep spindles in a defined amount of time. It may e.g. be measured as the number of detected sleep spindles per minute. For a subject to be classified as having an increased risk of developing a synucleinopathy, the sleep spindle density may for example be decreased by at least a factor 0.9, such as at least by a factor 0.8, for example at least by a factor 0.7, such as at least by a factor 0.6.

In a preferred embodiment, sleep spindles are detected in EEG recordings derived from one or more non-REM (NREM) sleep stages, such as from one or more of N1, N2, N3 or all NREM sleep stages combined.

Early identification of patients having an increased risk of developing a synucleinopathy allows for earlier treatment of the subject. It has been proposed that the efficiency of treatment is better if treatment is initiated as early as possible. Thus in one embodiment, the invention relates to medicinal or other treatment of a subject who has been identified as having an increased risk of developing a synucleinopathy. The specific treatment depends on the particular disease and can be determined by the skilled person.

### Parkinson's Disease

The pathology of PD is complex and not fully understood. It is characterized by the accumulation of Lewy bodies in neurons, and from insufficient formation and activity of dopamine produced in certain neurons within parts of the midbrain. Lewy bodies are the pathological hallmark of the idiopathic disorder, and the distribution of the Lewy bodies throughout the Parkinsonian brain varies from one individual to another. The anatomical distribution of the Lewy bodies is often directly related to the expression and degree of the clinical symptoms of each individual.

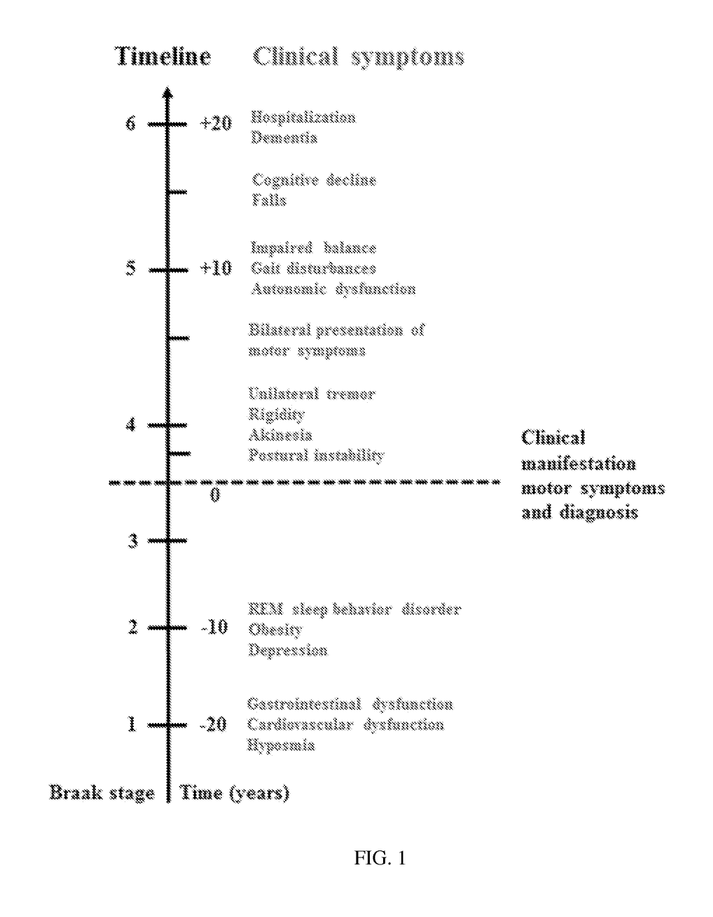

The pathology of PD can be described by the Braak stage model, which classifies the degree of pathology into one of six Braak stages. A simplified overview of the pathological process and the clinical symptoms is shown in FIG. 1. The first area to be affected is the brain stem, in particular the lower brainstem, i.e. the medulla oblongata. The medulla oblongata is affected in Braak stage 1 and correlates with symptoms of gastrointestinal dysfunction, cardiovascular dysfunction and/or hyposmia. The whole brain stem is affected in Braak stage 2. In Braak stage 2, symptoms like REM sleep behaviour disorder, obesity and/or depression appear. The midbrain becomes affected in Braak stage 3 and the classical motor symptoms of PD start to appear. The areas of the brain affected by the disease reflect the symptoms experienced by the patient. Thus, PD is a result of progressive destruction of neurons in the brain. The basal ganglia, which are innervated by the dopaminergic system, are the most seriously affected brain areas in PD. The main pathological characteristic of PD is cell death in the substantia nigra and, more specifically, the ventral part of the pars compacta, affecting up to 70% of the cells by the time death occurs.

When the diagnosis is made based on the manifestation of the motor symptoms of the disease, the brain is already severely affected as the motor symptoms of PD arise from the loss of dopamine-generating neurons in the substantia nigra of the midbrain.

In a preferred embodiment, the method of the present invention is performed before the clinical manifestation of motor symptoms of PD including tremor, rigidity, akinesia and postural instability. Clinical onset of PD is herein defined as the point in time when the above-mentioned motor symptoms are able to be diagnosed by a medical professional. Thus, the method of the present invention is preferably performed before substantial neurodegeneration in the midbrain has taken place, i.e. before the disease progresses to the midbrain.

In one embodiment the subject of the present invention suffers from one or more of the following symptoms preceding clinical manifestation of PD with approximately 10 to 20 years: Gastrointestinal dysfunction, cardiovascular dysfunction, hyposmia, RBD, obesity and/or depression. Preferably, the subject suffers from one or more of RBD, obesity and/or depression.

In one embodiment, the invention relates to medicinal or other treatment of a subject who has been identified as having an increased risk of developing Parkinson's disease. For instance, a patient identified as having an increased risk of developing Parkinson's disease could be administered PD drugs such as levodopa, dopamine agonists and MAO-B inhibitors before motor symptoms set in. A patient predicted to have an increased risk of developing PD according to the present invention may also be treated with e.g. a PD vaccine. Such early treatment could potentially inhibit or at least delay disease progression significantly.

### Multiple-System Atrophy

Multiple-system atrophy (MSA) is a degenerative neurological disorder. MSA is associated with the degeneration of nerve cells in specific areas of the brain. This cell degeneration causes problems with movement, balance, and other autonomic functions of the body such as bladder control or blood-pressure regulation.

In one embodiment, the method of the present invention relates to identification of subjects having an increased risk of developing MSA. Preferably, the subject is identified before clinical onset of the disease, i.e. before the point in time when MSA can be diagnosed by a medical professional.

Dementia with Lewy Bodies

Dementia with Lewy bodies (DLB), also known under a variety of other names including Lewy body dementia, diffuse Lewy body disease, cortical Lewy body disease, and senile dementia of Lewy type, is a type of dementia closely associated with both Alzheimer's and Parkinson's diseases. It is characterized anatomically by the presence of Lewy bodies, clumps of alpha-synuclein and ubiquitin protein in neurons, detectable in post mortem brain histology. Dementia with Lewy bodies overlaps clinically with Alzheimer's disease and Parkinson's disease, but is more associated with the latter. In DLB, loss of cholinergic neurons is thought to account for degeneration of cognitive function (similar to Alzheimer's), while the death of dopaminergic neurons appears to be responsible for degeneration of motor control (similar to Parkinson's)—in some ways, therefore, it resembles both diseases.

In one embodiment the method of the present invention relates to identification of subjects having an increased risk of developing DLB. In one embodiment, the method of the present invention relates to identification of subjects having an increased risk of developing DLB. Preferably, the subject is identified before clinical onset of the disease, i.e. before the point in time when DLB can be diagnosed by a medical professional.

### Sleep Spindle Biomarker

The sleep spindle biomarker of the present invention is based on automatic detection of sleep spindles in polysomnographic recordings. Manual scoring of sleep spindles is performed by sleep experts and is extremely time-consuming. Hence it is a great advantage to use an automatic sleep spindle detector capable of detecting sleep spindles with accuracy comparable to or even exceeding that of manual scoring.

In one embodiment, the present invention therefore relates to a computer implemented method for detecting sleep spindles in one or more EEG derivations acquired from a sleeping subject, the method comprising
a) dividing each EEG derivation into a plurality of time segments,
b) processing each time segment by means of a matching pursuit algorithm, such as Mallat & Zhang, providing Gabor atoms and the energy density of each time segment,
c) calculating a plurality of predefined features for each time segment, said features selected from the group of:
   energy features representing the energy density in each of a plurality of frequency bands,
   energy contribution features representing the energy contribution of at least one Gabor atom, preferably the first Gabor atom, in one or more of said frequency bands,
   a maximum energy feature representing the maximum energy point in the energy density, and
   the frequency corresponding to the maximum energy point in the energy density, and
d) based on said features classifying each time segment as 1) comprising a sleep spindle or at least a part of a sleep spindles, or 2) a background signal.

As stated previously, sleep spindles are bursts of oscillatory brain activity during non-REM (NREM) sleep, typically bursts of synchronous alpha waves. They can be seen as transient waveforms in electroencephalogram (EEG) derivations acquired from sleeping subjects. However, not all sleep spindles can be seen by the naked eye and the advantage of the present automatic sleep spindle detector is not only the speed and ease of detection but also the ability to detect sleep spindles that are “hidden” in the signal and thereby impossible to detect and characterize manually.

Traditionally, SS have been defined as nearly sinusoidal waves with a frequency profile at 12-14 Hz lasting at least 0.5 seconds and displaying an increasing, then decreasing amplitude envelope. This definition has later expanded to include frequencies in the range 12-16 Hz. The current AASM standard has expanded the frequency range to 11-16 Hz. However, the current AASM standard also imposes the restriction that a sleep spindle must be manually detectable. Thus, as used herein a sleep spindle is defined as a burst of oscillatory brain activity during non-REM sleep.

In one embodiment of the invention a sleep spindle is defined as a burst of oscillatory brain activity with the corresponding EEG signal comprising sinusoidal or nearly sinusoidal waves. A sleep spindle may be defined in a predefined frequency range and/or with a predefined minimum and/or maximum duration. A sleep spindle may further be characterized by a progressively increasing, then gradually decreasing amplitude. A sleep spindle may further be characterized as one or more groups of rhythmic waves. Thus, a sleep spindle may be defined as a short sinusoid event of duration 0.5-3 seconds with a frequency of 11-16 Hz.

Sleep spindles may be further classified into two categories: Slow sleep spindles and fast sleep spindles where the separation between the two SS categories is defined by a frequency, typically around 14 Hz. Thus, slow SS may be defined as comprising frequencies of 11.5-14 Hz and fast SS with frequencies of 14-16 Hz.

In a further embodiment of the invention a sleep spindle is defined according to the AASM standard.

In the field of pattern recognition, feature extraction or feature selection refers to the selection of variables that can differentiate between classes. When detecting sleep spindles the problem is a two-class problem, in which the SS make up one class and the background EEG make up the other class. The Matching Pursuit (MP) algorithm has been chosen for the feature extraction in the classification of sleep spindles. By decomposing a signal into basic waveforms, a detailed, reliable and sensitive parameterization is performed. The waveforms hold the following parameters: time position, frequency and duration, and by adjusting these, SS descriptors can be achieved.

Matching Pursuit (MP) is a signal processing algorithm, which was developed by Mallat and Zhang (Mallat and Zhang 1993; Mallat and Zhang 2008). The concept of MP is similar to traditionally decomposition methods, in which a given signal is represented by a sum of known basic waveforms, mathematically expressed as

$$f ( t ) = {\sum }_{n = 1}^{N} {a}_{n} {g}_{n} ( t )$$

Here, f is the original signal to be analysed, gₙ is the known basic waveform used to describe the signal, and aₙ is the weighting of each basic waveform. This equation is theoretical and not practical, as it states that N functions can represent the signal exactly.

In a wavelet transform a signal is decomposed using not only one particular function (as sinusoids in Fourier), but a family of functions called wavelets. A wavelet function is an oscillating function with compact support and with an amplitude that starts out at zero. In that way, a time resolution is achieved. The sinusoids used in the Fourier transform, and the wavelets used in the Wavelet transform are called dictionaries. In the Fourier transform and in a Wavelet transform these basis functions are orthogonal, and thereby give a unique decomposition of the signal. The idea behind the matching pursuit algorithm is to construct a dictionary so rich, that it can fit all possible structures of any signal of interest. This extension of limits is achieved by constructing the dictionary by Gabor functions. A Gabor function gives a frequency decomposition like the Fourier transform and a time resolution like a Wavelet transform. In the case of MP the Gabor functions are referred to as Gabor atoms. Gabor atoms are constructed by multiplying Gaussian envelopes and sinusoids. Giving a fixed time window, the Gaussian envelopes can vary by their time width and the position of the center and the sinusoids can vary by their frequency and phase. In this way, a Gabor atom has four adjustable parameters which combined can yield a wide variety of structures.

Mathematically a Gabor atom can be described as.

$${g}_{γ} ( t ) = K ( γ ) {e}^{- {π ( \frac{t - u}{s} )}^{2}} cos ( ω ( t - u ) + φ )$$

Here, γ={u, s, ω, φ} describes the adjustable parameters; the time-shift u, the width s, the frequency ω in rad/s and the phase φ in rad. K(γ) is a scaling factor. By adjusting the amplitudes of the Gabor atoms in the dictionary so that each function has unit energy (the parameter K(γ)), the product of one Gabor atom with the analysed signal will directly measure the contribution of that specific Gabor atom to the energy of the signal.

Several available Gabor atoms have overlap between them, and because of this, several similar atoms will fit the analysed signal. If taking out all atoms having high correlation with the signal, the resulting representation will contain many similar waveforms, all approximating only the strongest structure of the analysed signal. In concordance with the redundancy issue, the MP decomposition must therefore follow an iterative process, where the best choice of Gabor atom is found and then subtracted from the analysed signal before the next best match is found and so forth. In this way, only the chosen atoms from the redundant dictionary Dy are used to approximate the analysed signal. The original signal f is decomposed into a sum of dictionary elements (Gabor atoms), that are chosen to best match its residues.

In MP, it is not known a priori which Gabor atoms will be chosen. Because of this and the fact that the MP decomposition is an adaptive process, it is not possible to draw a prior division of the time-frequency distribution of energy density as it is in the case of the Wavelet transform and the short time Fourier. Mallat and Zhang presented a way of conducting the time-frequency energy distribution of a signal decomposed by MP by use of the Wigner Ville transform. The energy density of the signal can then be described as

$${E}_{f} ( t , ω ) = {\sum }_{n = 0}^{M - 1} {\vert 〈 {R}^{n} f ( t ) , {g}_{{γ}_{n}} ( t ) 〉 \vert}^{2} {WV}_{{g}_{{γ}_{n}}} ( t , ω ) .$$

In the development of a successful SS classifier, the feature selection and extraction are essential to obtain good performance. The features must reflect properties about the SS and be able to discriminate between SS and the background EEG signal. It is therefore an advantage to select several features, where some reflect different properties about the SS and others reflect different properties about the background EEG.

Each feature value can be calculated from each time segment, e.g. time segment as a two second long extract of the signal. The extracts are advantageously provided with overlap, e.g. with an overlap of one second. Time segments of 2 seconds and overlaps of 1 second may advantageously be selected because most sleep spindles have durations of between one and two seconds.

The first feature group is the energy features representing energy parts in a plurality of frequency bands. The energy feature values may be the energy E in one of these frequency bands normalised by the total energy Eₜₒₜₐₗ over all the frequencies. The plurality of frequency bands may comprise a lower frequency band, an upper frequency band and one or more sleep spindle frequency bands between said lower and upper bands. E.g. a sleep spindle frequency band may be between 11 and 16 Hz or between 12 and 16 Hz or between 11 and 14 Hz or between 11.5 and 14 Hz or between 11.5 and 14.5 Hz or between 14 and 16 Hz. The first frequency band may hold frequencies below 11 Hz, the second band may hold SS frequencies of 11-16 Hz and the third band may hold frequencies above 16 Hz. The energy E in a frequency band can be determined by taking the Gabor atoms with the respective frequencies in this band and compute the energy density maps by using the equation above by discrete integrating over time and frequency.

The second feature group relates to the total number of Gabor atoms in each frequency band. Typically the more complex a signal is more Gabor atoms are needed to represent the signal. A sleep spindle SS may be characterized as a progressively increasing and then gradually decreasing amplitude. As a Gabor function is a sinusoid with such an envelope, there may be a high correlation between a sleep spindle and a Gabor atom. Therefore, if a SS is present in a time segment, there might be very few Gabor atoms with frequencies in a sleep spindle frequency band.

The third feature group relate to the energy contribution of Gabor atoms, preferably the first Gabor atom, in the sleep spindle frequency band(s). All atoms with frequencies within each sleep spindle frequency band can be found and preferably the first one, hence the one with the lowest atom number, is taken out. This first Gabor atom is typically the one with the highest correlation with the signal, and hence a high energy contribution from this Gabor atom should indicate high SS activity. The logarithm of this energy contribution may advantageously be used as a normalization factor.

The fourth feature group relates to the maximum energy and the point where it is located, thus a maximum energy feature representing the maximum energy point in the energy density and the frequency corresponding to the maximum energy point in the energy density. The logarithm of this maximum energy may advantageously be used as a normalization factor. In general normalization and/or scaling of all the features may advantageously be provided.

An EEG measurement is normally acquired from a number of positions at the head of the subject providing a number of EEG derivations, each derivation corresponding to a specific position on the scalp. When detecting sleep spindles two, three or four EEG derivations are typically used and they are analysed concurrently. Thus, six feature values may be selected for each EEG derivation. With e.g. three EEG derivations the total number of features become 18, i.e. 18 feature values are calculated for each time segment. Each time segment with corresponding feature values is subsequently classified to comprise a sleep spindle (or at least a part of a sleep spindle) or be a background EEG signal. The classification can advantageously be provided by means of the Support Vector Machine (SVM) algorithm, see example 1.

In one embodiment, the energy contribution feature is calculated as the logarithm of the energy contribution of at least one Gabor atom in a predefined frequency band.

In one embodiment, a maximum energy feature is calculated as the logarithm of the maximum energy point.

In one embodiment, the energy features are normalized with the total energy density.

In one embodiment, the plurality of frequency bands comprise a lower frequency band, an upper frequency band and one or more sleep spindle frequency bands between said lower and upper band.

In one embodiment, a sleep spindle frequency band is between 11 and 16 Hz or between 12 and 16 Hz.

In one embodiment, a sleep spindle frequency band is between 11 and 14 Hz or between 11.5 and 14 Hz or 11.5 and 14.5 Hz.

In one embodiment, a sleep spindle frequency band is between 14 and 16 Hz.

In one embodiment, the energy contribution features representing the energy contribution of at least one Gabor atom is calculated in said one or more sleep spindle frequency bands between said lower and upper bands.

In one embodiment, the computer implemented method of the present invention further comprises the step of band pass filtering the EEG derivations prior to signal processing, such as band pass filtering from 2 to 35 Hz.

In one embodiment, the time segments are overlapping, such as overlapping by a number or seconds, such between 0.5 and 5 seconds, such as 1 second, or 2 seconds, or 3 seconds.

In one embodiment, each time segment corresponds to a number of seconds, such between 1 and 10 seconds, or between 1 and 2 second, or between 2 and 3 seconds, or between 3 and 5 second, or between 5 and 10 second, preferably 2 seconds.

In one embodiment, a support vector machine (SVM) algorithm is applied for classifying the time segments.

In one embodiment the early diagnosis of synucleinopathies according to the present invention comprises use of the computer implemented method as described herein above for detecting sleep spindles described herein above.

Combination with Further Biomarkers

The sleep spindle biomarker of the present invention may be combined with measurements of one or more further biomarkers, such as one or more further biomarkers derived from one or more polysomnographic recordings, in particular a biomarker based on automatic analysis of abnormal motor activity during REM sleep, a biomarker based on automatic analysis of electrooculography (EOG) signals and/or a biomarker based on automatic analysis of autonomic dysfunction.

Combination with other biomarkers can increase the sensitivity and specificity of the diagnostic method as described above.

### Biomarker Based on Automatic Analysis of Abnormal Motor Activity During REM Sleep

In one embodiment, the sleep spindle biomarker of the present invention is measured in combination with a biomarker based on automatic analysis of abnormal motor activity during REM sleep.

The automatic analysis of abnormal motor activity during REM sleep is performed essentially as described in EP12171637 filed 12 Jun. 2012, which is hereby incorporated by reference in its entirety. Automatic analysis of abnormal motor activity during REM sleep may also be performed essentially as described by Kempfner et al. in Kempfner et al., 2012a; Kempfner et al., 2012b; and Kempfner et al., 2011, all of which are which are hereby incorporated by reference in their entirety.

In one embodiment the biomarker based on automatic analysis of abnormal motor activity during REM sleep is determined according to a method comprising the following steps:
a. performing polysomnographic recordings of a sleeping subject thereby obtaining one or more EEG derivations, one or more electrooculargraphy (EOG) derivations and one or more electromyography (EMG) derivations,
b. detecting one or more REM sleep stages based on the one or more EEG and EOG derivations,
c. determining the level of muscle activity during the one or more REM sleep stages based on the one or more EMG derivations,
wherein a subject having an increased level of muscle activity during REM sleep compared to one or more normal subjects has an increased risk of developing a synucleinopathy.

Preferably, the above method is a computer implemented method which does not require manual analysis of the polysomnographic recordings.

### Biomarker Based on Automatic Analysis of Electrooculography (EOG) Signals

In one embodiment, the sleep spindle biomarker of the present invention is measured in combination with a biomarker based on automatic analysis of electrooculography (EOG) signals.

Automatic analysis of electrooculography (EOG) signals may be performed essentially as described in EP12181048 filed 20 Aug. 2012, which is hereby incorporated by reference in its entirety. Automatic analysis of electrooculography (EOG) signals may be also be performed essentially as described in Christensen et al. (2012).

In one embodiment the biomarker based on automatic analysis of EOG signals is determined according to a method comprising the following steps:
a. performing polysomnographic recordings of a sleeping subject thereby obtaining one or more EOG derivations,
b. determining the morphology and distribution of eye movements in the one or more EOG derivations,
wherein a subject having an altered morphology and/or distribution of eye movements compared to one or more normal subjects has an increased risk of developing a synucleinopathy.

Preferably, the above method is a computer implemented method which does not require manual analysis of the polysomnographic recordings.

### Biomarker Based on Automatic Analysis of Autonomic Dysfunction

In one embodiment, the sleep spindle biomarker of the present invention is measured in combination with a biomarker based on automatic analysis of autonomic dysfunction.

The automatic analysis of autonomic dysfunction may be performed essentially as described in Sorensen et al., 2011, Sorensen et al., 2012a and Sorensen et al., 2012b, which are all hereby incorporated by reference in their entirety.

In one embodiment the biomarker based on automatic analysis of autonomic dysfunction is determined according to a method comprising the following steps:
a. performing polysomnographic recordings of a sleeping subject thereby obtaining one or more EEG derivations and one or more electrocardiogram (ECG) derivations,
b. detecting arousals in the one or more EEG derivations,
c. determining the pulse response in connection with the arousals using the one or more ECG derivations,
wherein a subject having an altered pulse response in connection with arousals compared to one or more normal subjects has an increased risk of developing a synucleinopathy.

In an alternative embodiment the biomarker based on automatic analysis of autonomic dysfunction is determined according to a method comprising the following steps:
a. performing polysomnographic recordings of a sleeping subject thereby obtaining one or more EMG derivations and one or more electrocardiogram (ECG) derivations,
b. detecting motor activity in the one or more EMG derivations,
c. determining the pulse response in connection with the motor activity using the one or more ECG derivations,
wherein a subject having an altered pulse response in connection with arousals compared to one or more normal subjects has an increased risk of developing a synucleinopathy.

Preferably, the above methods are computer implemented methods which do not require manual analysis of the polysomnographic recordings.

### REFERENCES

Christensen J A E, Frandsen R, Kempfner J, Arvastson L, Christensen S R, Jennum P, Sorensen H B. Separation of Parkinson's patients in early and mature stages from control subjects using one EOG channel. Conf Proc IEEE Eng Med Biol Soc. 2012 Aug. 28, 2012.
Comella C L, Tanner C M, Ristanovic R K. Polysomnographic sleep measures in Parkinson's disease patients with treatment-induced hallucinations. Ann Neurol 1993; 34: 710-4.
Kempfner J, Jennum P, Nikolic M, Christensen J A E, Sorensen H B D. Automatic Detection of REM Sleep in Subjects without Atonia. Conf Proc IEEE Eng Med Biol Soc. 2012 Aug. 28, 2012.
Kempfner J, Sorensen G L, Nikolic M, Frandsen R, Sorensen H, Jennum P. Automatic detection of REM Sleep in recordings with muscle noise as present in patients with idiopathic REM behavioral disorder. Submitted to journal of Computer Methods and Programs in Biomedicine 2012.
Kempfner J, Sorensen G L, Sorensen H B, Jennum P. Automatic REM sleep detection associated with idiopathic rem sleep Behavior Disorder. Conf Proc IEEE Eng Med Biol Soc. 2011; 2011:6063-6.
Ktonas P Y, Golemati S, Xanthopoulos P, Sakkalis V, Ortigueira M D, Tsekou H et al. Time-frequency analysis methods to quantify the time-varying microstructure of sleep EEG spindles: possibility for dementia biomarkers? J Neurosci Methods 2009; 185: 133-42.
Mallat S G, Zhang Z, Franaszczuk P J, Jouny C C. [Matching Pursuit Software]. Aug. 21, 2008. Available at: http://erl.neuro.jhmi.edu/mpsoft/. Assessed May 16, 2012.
Mallat S G, Zhang Z. Matching pursuits with time-frequency dictionaries. IEEE Trans Signal Process 1993; 41: 3397-415.
Sorensen G L, Jennum P, Kempfner J, Zoetmulder M, Sorensen H B. A computerized algorithm for arousal detection in healthy adults and patients with Parkinson disease. J Clin Neurophysiol. 2012 February; 29 (1):58-64.
Sorensen G L, Kempfner J, Jennum P, Sorensen H B. Detection of arousals in Parkinson's disease patients. Conf Proc IEEE Eng Med Biol Soc. 2011; 2011:2764-7.
Sorensen G L, Kempfner J, Zoetmulder M, Sorensen H B, Jennum P. Attenuated heart rate response in REM sleep behavior disorder and Parkinson's disease. Mov Disord. 2012 June; 27(7):888-94.
Ventouras E M, Economou N T, Kritikou I, Tsekou H, Paparrigopoulos T J, Ktonas P Y. Performance evaluation of an artificial neural network automatic sleep spindle detection system. Conf Proc IEEE Eng Med Biol Soc. 2012: 4328-31.

### Example 1

### 1 ABSTRACT

Objective: To determine whether sleep spindles (SS) are potentially a biomarker for Parkinson's disease (PD). Methods: Fifteen PD patients with REM sleep behavior disorder (PD+RBD), 15 PD patients without RBD (PD-RBD), 15 idiopathic RBD (iRBD) patients and 15 age-matched controls underwent polysomnography (PSG). SS were scored in an extract of data from control subjects. An automatic SS detector using a Matching Pursuit (MP) algorithm and a Support Vector Machine (SVM) was developed and applied to the PSG recordings. The SS densities in N1, N2, N3, all NREM combined and REM sleep were obtained and evaluated across the groups. Results: The SS detector achieved a sensitivity of 84.7% and a specificity of 84.5%. At a significance level of α=1%, the iRBD and PD+RBD patients had a significantly lower SS density than the control group in N2, N3 and all NREM stages combined. At a significance level of α=5%, PD-RBD had a significantly lower SS density in N2 and all NREM stages combined. Conclusions: The lower SS density suggests involvement in pre-thalamic fibers involved in SS generation. SS density is a potential early PD biomarker.

### 2 METHODS

2.1 Subjects

Subjects were recruited from patients evaluated at the Danish Center for Sleep Medicine (DCSM) in the Department of Clinical Neurophysiology, Glostrup University Hospital. All patient evaluations included a comprehensive medical and medication history. All patients were assessed by polysomnography (PSG) and with a multiple sleep latency test (MSLT). Patients taking any anti-depressant drug, including hypnotics, were excluded, though dopaminergic treatments were continued. A total of 15 PD patients without RBD (PD−RBD), 15 PD patients with RBD (PD+RBD) and 15 iRBD patients were included. Fifteen age-matched control subjects with no history of movement disorder, dream-enacting behavior or other previously diagnosed sleep disorders were included. Patients using any type of medication known to affect sleep were also excluded. The demographic data for the three patient groups and the control group are summarized in table 1.

TABLE 1
Demographic data for the control and the patient groups.

| | | | | | Sleep | |
|---|---|---|---|---|---|---|
| | | Male/Female | Age | BMI | Efficiency | TRT |
| Patient Group | Frequency | frequency | [years] | [kg/m²] | [%] | [min] |
| Controls | 15 | 6/9 | 58.3 ± 9.5 | 23.2 ± 2.8 | 88.9 ± 8.4 | 480 ± 47.5 |
| iRBD | 15 | 12/3 | 60.1 ± 7.4 | 24.4 ± 3.1 | 85.6 ± 8.3 | 489 ± 95.3 |
| PD − RBD | 15 | 8/7 | 61.9 ± 6.1 | 24.7 ± 2.2 | 82.8 ± 7.9 | 443 ± 67.2 |
| PD + RBD | 15 | 11/4 | 62.4 ± 5.2 | 26.0 ± 3.2 | 85.4 ± 9.7 | 445 ± 71.8 |

2.2 Polysomnograph Recordings

Polysomnograph (PSG) data were collected in this study. All controls underwent at least one night of PSG recording as outpatients, and all patients underwent at least one night of PSG recording either as outpatients or in hospital in accordance with the AASM standard. When manually scoring the SS, only the F3-A2, C3-A2 and O1-A2 EEG derivations were visible for the SS scorer, and for 13 control subject a number of randomly selected sleep epochs, each of a duration of 30 seconds, were chosen for SS scoring. The selection of sleep epochs was carried out by the SS scorer, who aimed at selecting approximately 30 sleep epochs containing one or more visible SS randomly distributed across the sleep cycles. It was ensured that every SS within a chosen sleep epoch was marked. Filter conditions were as stated in the AASM standard, and the AASM standard SS definition was used, whereby SS have frequencies in the range 11-16 Hz, last for 0.5-3 seconds and have no amplitude criteria. The left EEG derivations were chosen as these are known to exhibit an overall higher spindle density. In order to reproduce realistic conditions, sleep epochs with moderate noise contamination were allowed and no artifacts were removed manually. The scoring yielded a total of 375 sleep epochs with 882 manually scored SS. The distribution of the chosen sleep epochs across the different sleep stages is seen in table 2. All the scored SS within these sleep epochs were confirmed by an expert. The raw sleep data, hypnograms and sleep events were extracted from Somnologica Studio (V5.1, Embla, Broomfield, Colo. 80021, USA) or Nervus (V5.5, Cephalon D K, Norresundby, Denmark), using the built-in export data tool. For further analysis, the data were imported into MATLAB (R2010b, MathWorks, Inc., Natick, Mass., USA).

TABLE 2
The distribution of the different sleep stages within the four groups evaluated
and for use in the development of the SS detector.

| | For use in the | | | | |
|---|---|---|---|---|---|
| | development | | | | |
| Sleep stage | of SS detector | Controls | iRBD | PD − RBD | PD + RBD |
| Wake (%) | 0 (0) | 1606 (11) | 2220 (15) | 2387 (18) | 1889 (14) |
| REM (%) | 4 (1) | 2710 (19) | 2893 (20) | 1808 (13) | 1761 (13) |
| N1 (%) | 13 (4) | 1205 (8) | 1238 (8) | 1191 (9) | 1623 (12) |
| N2 (%) | 330 (88) | 6491 (45) | 5909 (40) | 5817 (44) | 5957 (45) |
| N3 (%) | 28 (7) | 2388 (17) | 2423 (17) | 2097 (16) | 2128 (16) |
| Sum (%) | 375 (100) | 14400 (100) | 14683 (100) | 13300 (100) | 13358 (100) |

2.3 Development of SS Detector

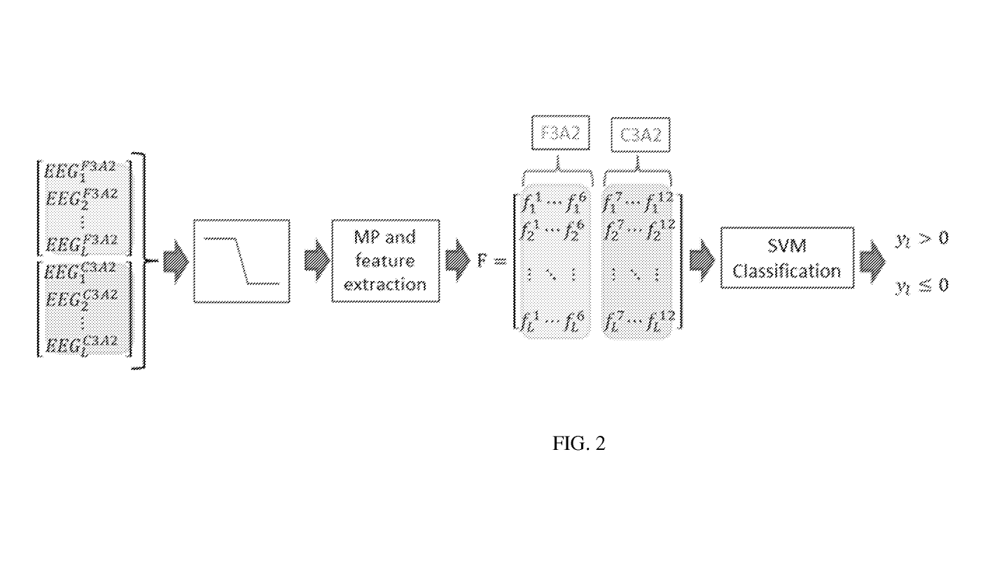

The steps in the method for developing the automatic detector are shown in FIG. 2. Firstly, appropriate features were extracted from the C3-A2 and F3-A2 EEG derivations. These are variables that represent characteristics of the classes and may therefore reflect differences between them. These were sent through a classifier that determines the class (‘SS’ or ‘background EEG’) to which the data segment belongs.

2.3.1 Feature Extraction

Before feature extraction, the polysomnograph C3-A2 and F3-A2 EEG derivations were band pass-filtered from 2 to 35 Hz. The lower cutoff frequency at 2 Hz was chosen to avoid the influence of the high-energy contents at the very low frequencies, and the cutoff at 35 Hz was chosen to reflect the AASM standard. The Matching Pursuit (MP) method was chosen for feature extraction in the classification of SS. In the MP signal processing algorithm a given signal is represented by a weighted sum of known basic waveforms, known as Gabor atoms, g_{γ}(t), which in continuous time are expressed as:

$${g}_{γ} ( t ) = K ( γ ) {e}^{- {π ( \frac{t - u}{s} )}^{2}} cos ( ω ( t - u ) + φ ) \qquad ( 1 )$$

Here, γ={u, s, ω, φ} represents time-shift u and width s in seconds, frequency ω in rad/s and the phase φ in rad. K(γ) is a normalization scaling factor. By making a redundant dictionary of Gabor atoms, the signal was decomposed iteratively, whereby the Gabor atom most highly correlated with the signal or its residual was chosen at each step. As the iterative process continues, the residual decays exponentially (Mallat and Zhang, 1993), and the process stops when the residual is below a given threshold. The MP algorithm projects a function f(t) on Gabor atoms:

$$f ( t ) = {\sum }_{n = 0}^{M - 1} 〈 {R}^{n} f ( t ) , {g}_{{γ}_{n}} ( t ) 〉 {g}_{{γ}_{n}} ( t ) + {R}^{M} f ( t ) \qquad ( 2 )$$

where

$${g}_{{γ}_{0}}$$

denotes the first selected atom, [symbol] Rⁿf(t),g_{γ}_{ₙ}(t) [symbol] the inner product of the atom and the signal Rⁿf(t) and R^{M}f(t) denotes the residual signal after approximating f(t) by using M Gabor atoms.
The time-frequency distribution of the signal energy is derived by adding Wigner-Ville distributions of selected atoms (Mallat and Zhang, 1993), which yields

$${WV}_{f} ( t , ω ) = {\sum }_{n = 0}^{M - 1} {\vert 〈 {R}^{n} f ( t ) , {g}_{{γ}_{n}} ( t ) 〉 \vert}^{2} {WV}_{{g}_{{γ}_{n}}} ( t , ω ) + {\sum }_{n = 0}^{M - 1} {\sum }_{k = 1 , k \neq n}^{M - 1} 〈 {R}^{n} f ( t ) , {g}_{{γ}_{n}} ( t ) 〉 〈 {R}^{k} f ( t ) , {g}_{{γ}_{k}} ( t ) 〉 {WV}_{{g}_{{γ}_{n}} , {g}_{{γ}_{k}}} ( t , ω ) , \qquad ( 3 )$$

where WV_{f} and

$${WV}^{{g}_{{γ}_{n}}}$$

indicate the Wigner-Ville distribution of the signal f and the given Gabor atom

$${}^{{g}_{{γ}_{n}}} ,$$

respectively. The first sum corresponds to the auto-terms and the double sum corresponds to the cross-terms of the Wigner-Ville transform. By removing the cross-terms, the energy density of the signal f(t) is found:

$${E}_{f} ( t , ω ) = {\sum }_{n = 0}^{M - 1} {\vert 〈 {R}^{n} f ( t ) , {g}_{{γ}_{n}} ( t ) 〉 \vert}^{2} {WV}_{{g}_{{γ}_{n}}} ( t , ω ) . \qquad ( 4 )$$

The features were all calculated from the energy densities derived from the Wigner-Ville transform. They were obtained from signal windows of 2 seconds with a 1-second overlap. For each EEG derivation, the features included:
1) Three energy features reflecting energy parts in the frequency bands f<11 Hz, 11 Hz≦f≦16 Hz and f>16 Hz, defining frequencies below, within and above the SS frequency band, respectively.
2) The logarithm of the energy contribution of the first Gabor atom with a frequency of 11 Hz≦f≦16 Hz.
3) The logarithm of the maximum energy point in the energy density found by equation (4) and the corresponding frequency.

The six feature values were calculated for the C3-A2 and F3-A2 EEG derivations, yielding a total of 12 feature values for each 2-second segment. The features were normalized with respect to the 95th percentile of the features, since this was the normalization method found to perform best.

2.3.2 Classification

In this study, the Support Vector Machine (SVM) algorithm was chosen to classify the SS. SVM is a binary supervised learning method, and has proved to be efficient when dealing with datasets of unequal size. Clearly, the essential goal in all machine learning techniques is to optimize the generalized classification properties of the model, i.e. to categorize correctly as many data points of an unseen dataset as possible. This optimization process is employed in the training phase, and the essence of SVM is to find optimal separating hyperplanes in a high-dimensional feature space. The optimization in SVM consists of maximising the margin between classes in the feature space, which is sometimes referred to as “the maximal margin classifier”.

A training dataset can mathematically be described as

{xᵢ,yᵢ}ᵢ₌₁^{L}yᵢε{−1,1}xᵢε [symbol] ^{D} (5)

where each of the L training samples xᵢ is a vector with D feature values and yᵢ takes the value of −1 or 1, indicating the group to which each training sample belongs. In the case of the two classes being linearly separable, they can be classified by a hyperplane described as

h(xᵢ)= [symbol] xᵢ,w [symbol] +b=0, (6)

where w is the normal to the hyperplane and b is a shifting constant. The finding of the hyperplane is based on the positive and negative samples of x(y₁ in FIG. 1) that are most strongly indicative of the slope of the resulting separating hyperplane. These are the support vectors, and they all satisfy the constraint:

yᵢ·( [symbol] xᵢ,w [symbol] +b)−1+ξᵢ≧0∀i, (7)

where ξᵢ≧0∀i is a slack variable introducing a cost or penalty to misclassified samples, relaxing the constraints of the fully linearly separable case. The penalty increases with the distance to the separating hyperplane.

To describe the separating hyperplane, the values for w and b are found by solving the problem summarized to:

$$\{ \begin{matrix}min ( \frac{1}{2} {‖ w ‖}^{2} + C {\sum }_{i = 1}^{L} {ξ}_{i} ) & \\ {y}_{i} ( 〈 {x}_{i} , w 〉 + b ) - 1 + {ξ}_{i} \geq 0 & \\ {ξ}_{i} \geq 0 & ∀ i \\ & ∀ i\end{matrix} \qquad ( 8 )$$

where the cost parameter C is a user-defined parameter indicating the penalty for misclassification. The problem is solved by introducing Lagrange multipliers, and knowing the values for w and b defines the optimal orientation of the separating hyperplane, and the SVM classifier is defined. The classification of a new unknown data point x′=[f¹ . . . f²] indicated by the 12 features described above merely requires the sign of the function

h(x′)= [symbol] x′,w [symbol] +b (9)

to be evaluated. The sign indicates on which side of the separating hyperplane the data point x′ lies.

The SVM classification can easily be extended to work on non-linear separable classes by using kernels K(xᵢ,xᵢ), mapping the data into a Euclidean space H where they can be linearly separated. In this study, a Radial Basis Function (RBF) kernel was used for the SVM, and a parameter optimization study was performed by doing a grid search on the cost parameter C and the kernel-specific parameter

$$γ = \frac{1}{2 {σ}^{2}} ,$$

which controls the flexibility of the decision boundaries with higher γ values allowing greater flexibility. The evaluated values were γ={0.125, 0.25, 0.5, 1, 2, 4} and C={1, 4, 16, 64, 256, 1024}. The optimal pair for the final model was found to be (C,γ)=(256,1).

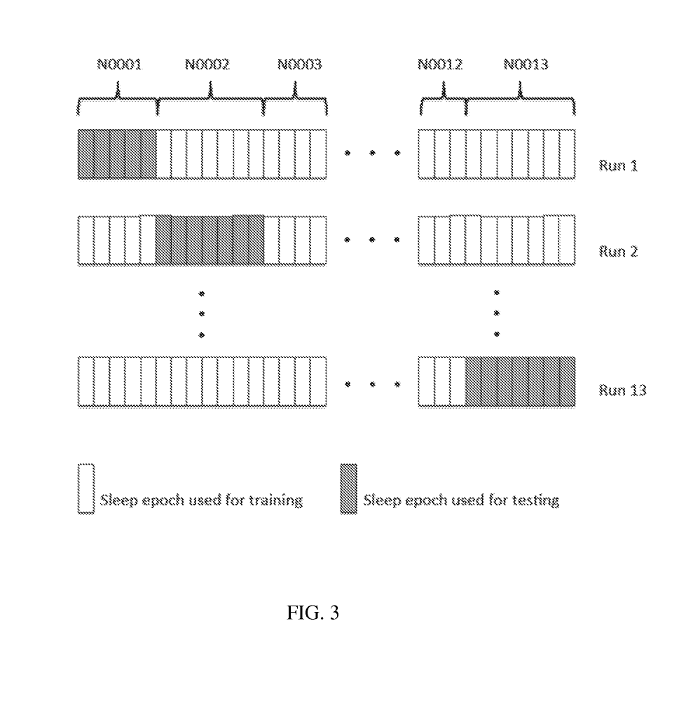

As in other studies, only the data with manually scored SS was used in the development of the automatic SS detector. Hence, the feature vectors from the sleep epochs with manual scores of SS were used to train and test the classifier in this study. Each second of EEG data was labeled either SS (1) or background EEG (−1). The training and testing phases employed the leave-one-subject-out strategy. As illustrated in FIG. 3, the test data set in each of the 13 runs were of unequal size, as the number of available scored sleep epochs differed between the control subjects. Overall performance measures were calculated as the mean of the 13 runs. The SVM^{perf} algorithm developed by Thorsten Joachims at Cornell University was used in this example.

### 3 RESULTS

3.1 Performance of Automatic SS Detector

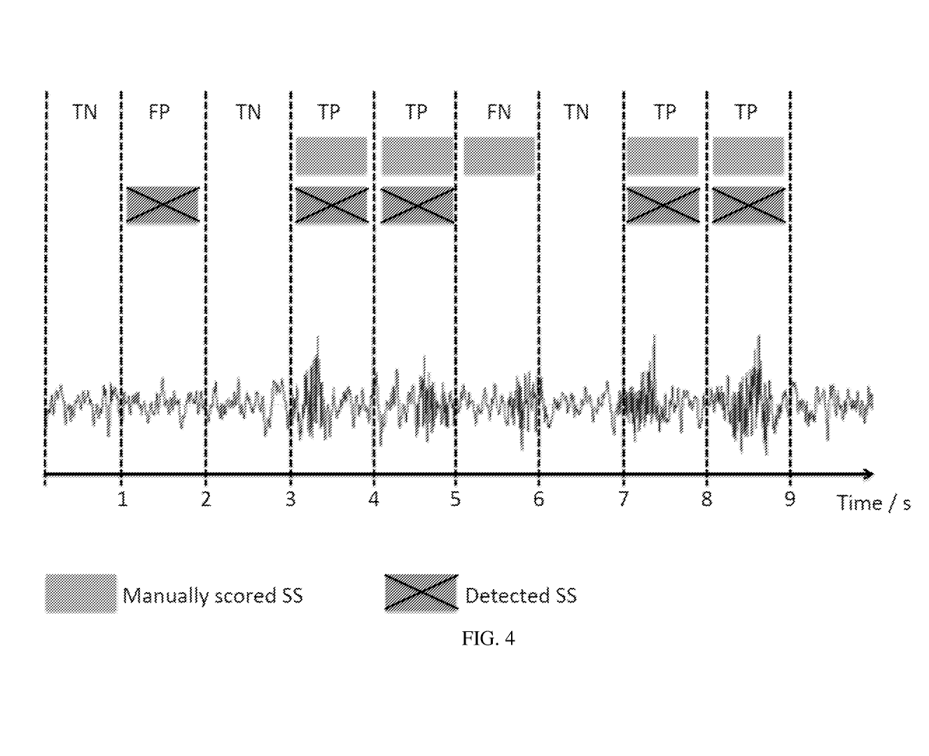

To validate the performance of the algorithm, different statistical measures were defined on the basis of four variables: True Positives (TP), False Positives (FP), True Negatives (TN) and False Negatives (FN). These were found by comparing the SS detected by the algorithm and those manually scored, as illustrated in FIG. 4.

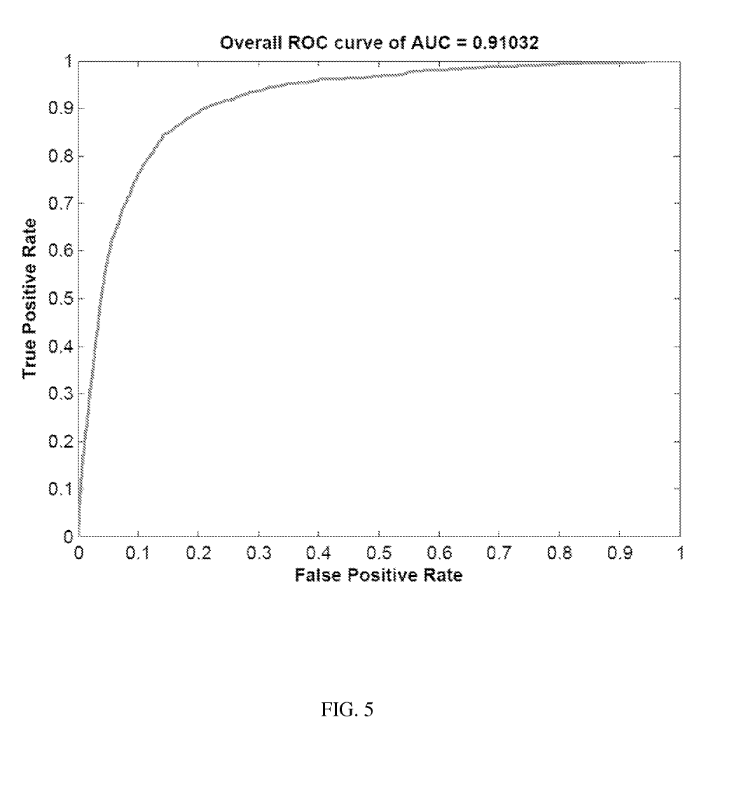

The values obtained were used to calculate the sensitivity and specificity, and by using these, a Receiver Operating Characteristics (ROC) curve was derived (FIG. 5). These values were obtained using the data with manually scored SS, i.e. the epochs stated under “For use in the development of SS detector” in table 2.

The area under the ROC curve (AUC) reached 91.0% based on the leave-one-subject-out strategy. By choosing the (FP, TP) pair as the point on the ROC curve, where the sign of the function described in equation (10) determined the class, the mean sensitivity reached 84.7% and the mean specificity reached 84.5%. These were considered satisfactory for the purpose of this study.

3.2 SS Densities

To determine whether the SS density varied between the three groups of patients and the control group, the automatic detector was applied to the all-night recordings from lights-off until lights-on. The total number and the distribution of the different sleep stages within the four groups are provided in table 2. SS density was defined as SS/min and measured for the different sleep stages. Specifically, sleep epochs of N1, N2, N3, all NREM and REM were evaluated separately. The values of the means and standard deviations of the various sleep stages and groups are shown in table 3.

TABLE 3
Means and standard deviations of the SS densities of the four groups
in the respective sleep stages. SS density was defined as SS/min.

| | | | | All | |
|---|---|---|---|---|---|
| Sleep stage | N1 | N2 | N3 | NREM | REM |
| Controls | 4.4 ± 1.6 | 6.2 ± 1.5 | 5.6 ± 1.3 | 6.0 ± 1.3 | 2.2 ± 1.4 |
| iRBD | 4.4 ± 1.7 | 4.7 ± 1.9 | 4.1 ± 2.4 | 4.5 ± 1.8 | 2.8 ± 1.4 |
| PD − RBD | 4.4 ± 1.7 | 5.1 ± 1.8 | 4.9 ± 2.3 | 5.0 ± 1.5 | 2.4 ± 1.4 |
| PD + RBD | 4.4 ± 2.1 | 4.2 ± 1.9 | 3.6 ± 2.1 | 4.2 ± 1.8 | 3.6 ± 2.2 |

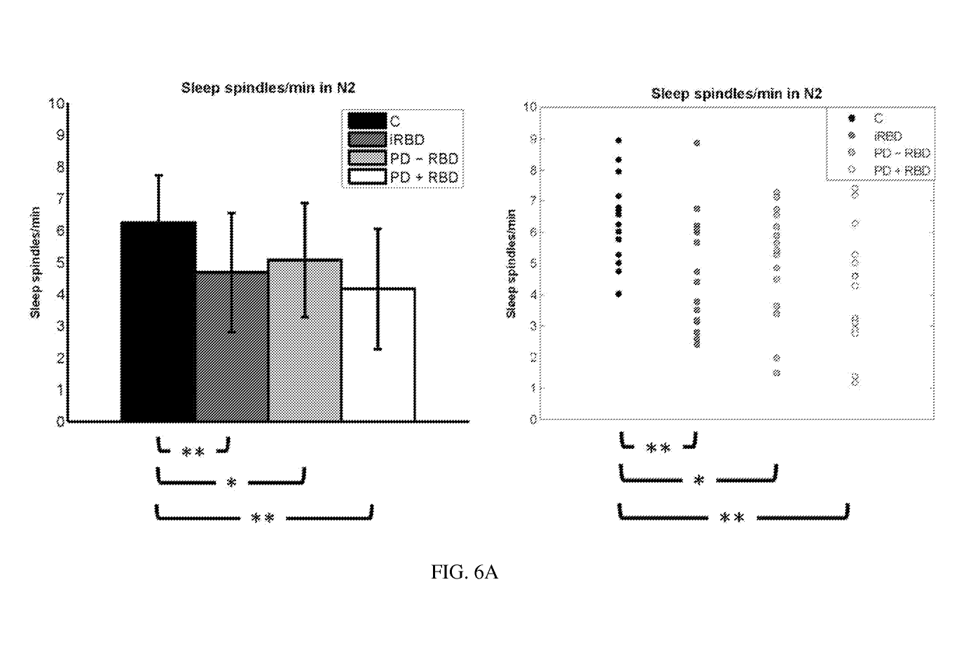

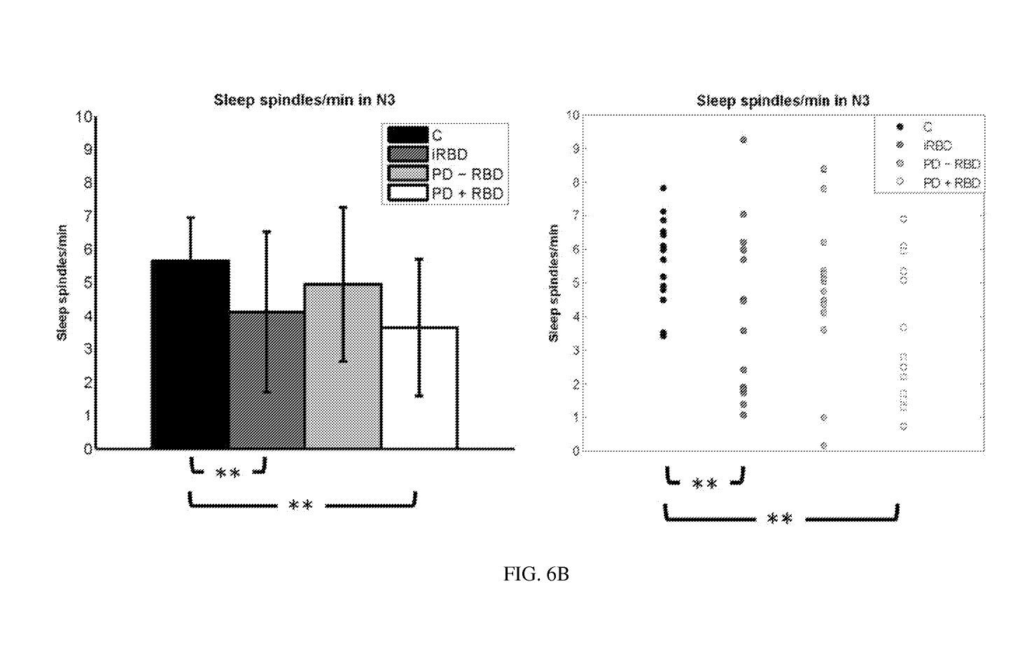

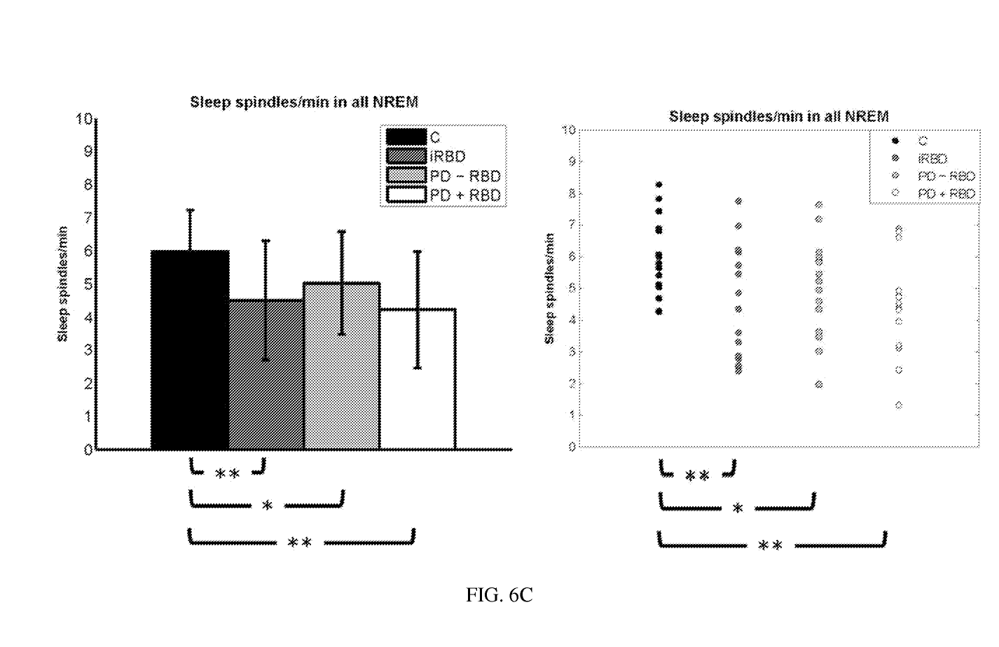

To establish whether there was a significant difference between the means of SS density in the four groups, unpaired two-sample t-tests were performed. The variances within each group were assumed to be unequal. Comparisons of the control group with a diseased group used one-sided t-tests, whereas those of pairs of diseased groups used two-sided tests. In this way, it was established whether the mean of each diseased group was lower than that of the control group, and whether the means of the diseased groups differed from one another. The significant differences are illustrated in FIG. 6. At a significance level of α=1%, the iRBD and PD patients with RBD had a significantly lower mean SS density than the control group in N2, N3 and all NREM combined. At a significance level of α=5%, the PD patients without RBD had a significantly lower mean SS density than the control group in N2 and all NREM combined.

### 4 CONCLUSION

The study develops a novel approach for designing an automatic SS detector. Applying this detector to data from iRBD and PD patients as well as age-matched controls, SS densities were obtained from different sleep stages and proved to be significantly lower for the iRBD group and the PD groups with and without RBD compared with the controls in NREM sleep. The lower SS density suggests involvement in pre-thalamic fibers involved in SS generation. We conclude that SS is a potential biomarker for early detection of PD, and it is likely that an automatic SS detector could be a diagnostic tool for identifying subjects having an increased risk of developing PD and other synucleinopathies.

### INCORPORATION BY REFERENCE OF PRIOR APPLICATION

This application claims priority under 35 U.S.C. §119 or 365 to European Application No. 13169679.1, filed May 29, 2013, the entire teachings of which are incorporated herein by reference.

1. A method for identifying a subject having an increased risk of developing a synucleinopathy comprising detection of sleep spindles.

2. The method according to claim 1, wherein the subject is identified before clinical onset of the synucleinopathy.

3. The method according to claim 1, wherein the method comprises the steps of:
a) acquiring one or more electroencephalographic (EEG) derivations from a sleeping subject;
b) detecting sleep spindles in said one or more EEG derivations; and
c) determining the density of sleep spindles in said one or more EEG derivations,
wherein a subject having a decreased sleep spindle density has an increased risk of developing a synucleinopathy.

4. The method according to claim 3, wherein the one or more EEG derivations are derived from one or more non-rapid eye movement (NREM) sleep stages.

5. The method according to claim 3, wherein the detection and determination of sleep spindle density is fully automated.

6. The method according to claim 3, wherein the detection and determination of sleep spindle density does not involve manual analysis of the EEG derivations by a sleep expert.

7. The method according to claim 3, wherein the decreased sleep spindle density is in comparison to the sleep spindle density in a group of healthy subjects.

8. The method according to claim 3, wherein the decreased sleep spindle density is in comparison to a previous measurement of sleep spindle density in the same subject.

9. The method according to claim 3, wherein the method further comprises detection of one or more further biomarkers.

10. The method according to claim 3, wherein the one or more further biomarkers are derived from one or more polysomnographic recordings.

11. The method according to claim 3, wherein the one or more further biomarkers are selected from automatic analysis of abnormal motor activity during REM sleep, automatic analysis of electrooculography (EOG) signals or automatic analysis of autonomic dysfunction.

12. The method according to claim 1, wherein the synucleinopathy is selected from Parkinson's disease, Multiple System Atrophy or Dementia with Lewy Bodies.

13. The method according to claim 12, wherein the synucleinopathy is Parkinson's disease.

14. The method according to claim 13, wherein the subject is identified before manifestation of one or more motor symptoms selected from tremor, rigidity, akinesia or postural instability.

15. The method according to claim 1, wherein the subject is identified before substantial neurodegeneration has occurred.

16. The method according to claim 1, wherein the method is a computer implemented method.

17. The method according to claim 1, wherein the detection of sleep spindles is performed by a computer implemented method for detecting sleep spindles in one or more electroencephalographic (EEG) derivations acquired from a sleeping subject, the method comprising;
a) dividing each EEG derivation into a plurality of time segments;
b) processing each time segment by means of a matching pursuit algorithm, providing Gabor atoms and the energy density of each time segment; and
c) calculating a plurality of predefined features for each time segment, said features selected from;
energy features representing the energy density in each of a plurality of frequency bands;
energy contribution features representing the energy contribution of at least one Gabor atom, preferably the first Gabor atom, in one or more of said frequency bands,
a maximum energy feature representing the maximum energy point in the energy density, and
the frequency corresponding to the maximum energy point in the energy density, and
based on said features classifying each time segment as 1) comprising a sleep spindle or at least a part of a sleep spindles, or 2) a background signal.

18. A computer implemented method for detecting sleep spindles in one or more EEG derivations acquired from a sleeping subject, the method comprising
a) dividing each electroencephalographic (EEG) derivation into a plurality of time segments;
b) processing each time segment by means of a matching pursuit algorithm, providing Gabor atoms and the energy density of each time segment; and
c) calculating a plurality of predefined features for each time segment, said features selected from;
energy features representing the energy density in each of a plurality of frequency bands,
energy contribution features representing the energy contribution of at least one Gabor atom, preferably the first Gabor atom, in one or more of said frequency bands,
a maximum energy feature representing the maximum energy point in the energy density, and
the frequency corresponding to the maximum energy point in the energy density, and
based on said features classifying each time segment as 1) comprising a sleep spindle or at least a part of a sleep spindles, or 2) a background signal.
